(19) United States
(12) Patent Application Publication
(10) Pub. No.: US 2013/0011566 A1
(43) Pub. Date: Jan. 10, 2013

(54) Petri Dish Comprising Means for Holding Agar Culture Medium

(75) Inventors: Bruno Colin, Marcy I'Etoile (FR); Nathalie Simon, Grezieu La Varenne (FR)

(73) Assignee: Bio Merieux ,SA, Marcy L'Etoile (FR)

(21) Appl. No.: 13/636,196

(22) PCT Filed: Mar. 24, 2011
(86) PCT No.: PCT/FR2011/050625

§ 371 Date: Sep. 20, 2012

(30) Foreign Application Priority Data
Mar. 24, 2010 (FR) 1052121

(51) Int. Cl. C12M 1/22; B05D 3/00

(52) U.S. Cl. 427/398.1; 435/305.4

(57) ABSTRACT

The present invention relates to a Petri dish, intended for receiving an agar culture medium for microorganisms, including a base receiving said culture medium and a lid capable of being positioned on the base, said base and said lid being substantially circular, said Petri dish being characterised in that the base includes at least one means intended to avoid the retraction of the culture medium, said means projecting and being positioned on the periphery of the base.

The technical field of the invention is that of in vitro diagnosis and more precisely, that of microbiological diagnosis. The present invention relates in particular to a Petri dish presenting means for retaining the agar culture medium.

The use of Petri dishes in the microbiological field has been known for many decades. Indeed, these dishes are used as a container for an agar culture medium which is used to allow the growth and detection of the microorganisms. Petri dishes are generally circular and constituted of a bottom which contains the culture medium and a lid which is positioned on the bottom in order to avoid the medium being contaminated by microorganisms present in the external environment, whilst allowing the passage of air, which is necessary for the development of many microorganisms. There are two main forms of Petri dish:
the standard Petri dish, defined in the standard ISO 24988-2008, and
the Petri dish referred to as a “contact” Petri dish which makes it possible to carry out surface samplings and which has, for this purpose, a particular form such that the agar medium extends beyond the bottom.

Regardless of the type of dish, it plays an essential role in the quality and the performances of the culture medium as a tool for the detection and identification of microorganisms. Indeed, the dish must, in particular, display surfaces which are perfectly transparent and flawless, so as to avoid distorting the reading.

In spite of the high quality of the design of the Petri dish, with some agar culture media a retraction of the culture medium can occur once poured into the dish and solidified. This generally occurs after a certain storage time. This problem is well known to microbiologists and is directly linked to the composition of the culture medium. Indeed, certain compounds (such as the surface-active or lipid agents) present in the medium, substantially reduce the ability of said medium to adhere to the bottom of the dish into which it is poured. This is the case with the Ottaviani Agosti medium for example, which is used to search for and count Listeria monocytogenes, in accordance with the standard NF EN ISO 11290. Furthermore, the shape of Petri dishes, which is generally circular, is not conducive to keeping the agar properly in place. It follows that the agar medium firstly detaches from the vertical wall. There then occurs a phenomenon of radial retraction of the agar medium towards the centre of the Petri dish. Said agar medium ends up completely detaching from the Petri dish, at the risk of leaving the dish when the latter is handled.

An objective of the present invention is therefore to provide a circular Petri dish capable of limiting the effects of retraction of the agar culture medium contained in said dish.

Another objective of the present invention is therefore to provide a Petri dish presenting means intended to prevent the retraction of the agar culture medium, which do not impede the reading of the Petri dish.

These objectives amongst others are achieved by the present invention which firstly relates to a Petri dish, intended for receiving an agar medium for culture of microorganisms, including a base receiving said culture medium and a lid capable of being positioned on the base, said base and said lid being substantially circular, said Petri dish being characterised in that the base includes at least one means intended to avoid the retraction of the culture medium, said means projecting and being positioned on the periphery of the base.

According to a particular embodiment of the Petri dish according to the invention, the means intended to avoid the retraction of the culture medium is constituted by a continuous rib.

According to an alternative embodiment, the means intended to avoid the retraction of the culture medium is constituted by at least one non-continuous rib.

Advantageously, the Petri dish according to the invention includes means for locking the lid onto the base.

According to a preferred embodiment, the locking means include at least one radial fastening tab and at least one radial sheath, which are respectively positioned either on the lid, or on the base, and cooperate to lock the Petri dish.

Such locking means are described, for example, in U.S. Pat. No. 6,969,607.

Advantageously, the Petri dish according to the invention includes between 2 and 12 non-continuous ribs.

In particular, the means which is (are) intended to avoid the retraction of the culture medium has (have) a height of between 0.15 and 5 millimetres.

Said means which is (are) intended to avoid the retraction of the culture medium is (are) situated at a distance of between 0.5 and 15 millimetres from the circular vertical edge of the base.

Another object of the present invention relates to a method of preparing an agar culture medium including the steps consisting of:
possessing a Petri dish, according to the invention,
distributing a molten agar culture medium inside said Petri dish, such that it covers the entire bottom of the base,
allowing the agar culture medium to cool such that the means which is (are) intended to avoid the retraction are anchored in the agar culture medium.

The invention shall be better understood by reading the following detailed non-limiting description, with reference to the drawings, in which:

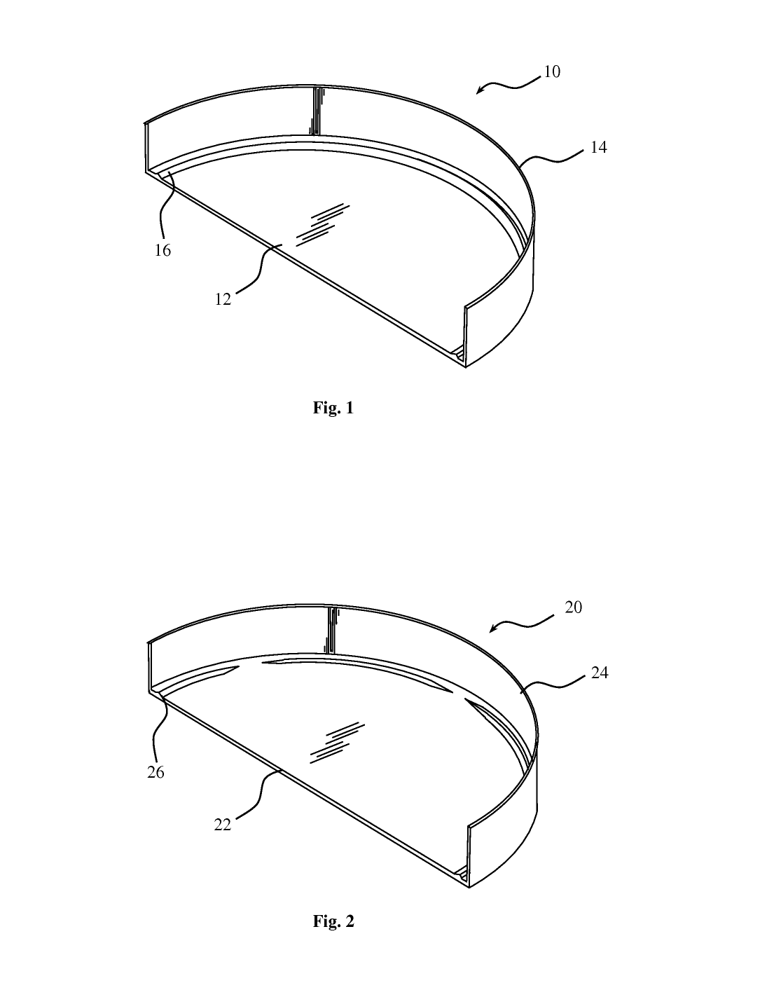

FIG. 1 shows a partial perspective view of the base of the Petri dish according to a first embodiment.

FIG. 2 shows a partial perspective view of the base of the Petri dish according to a second embodiment.

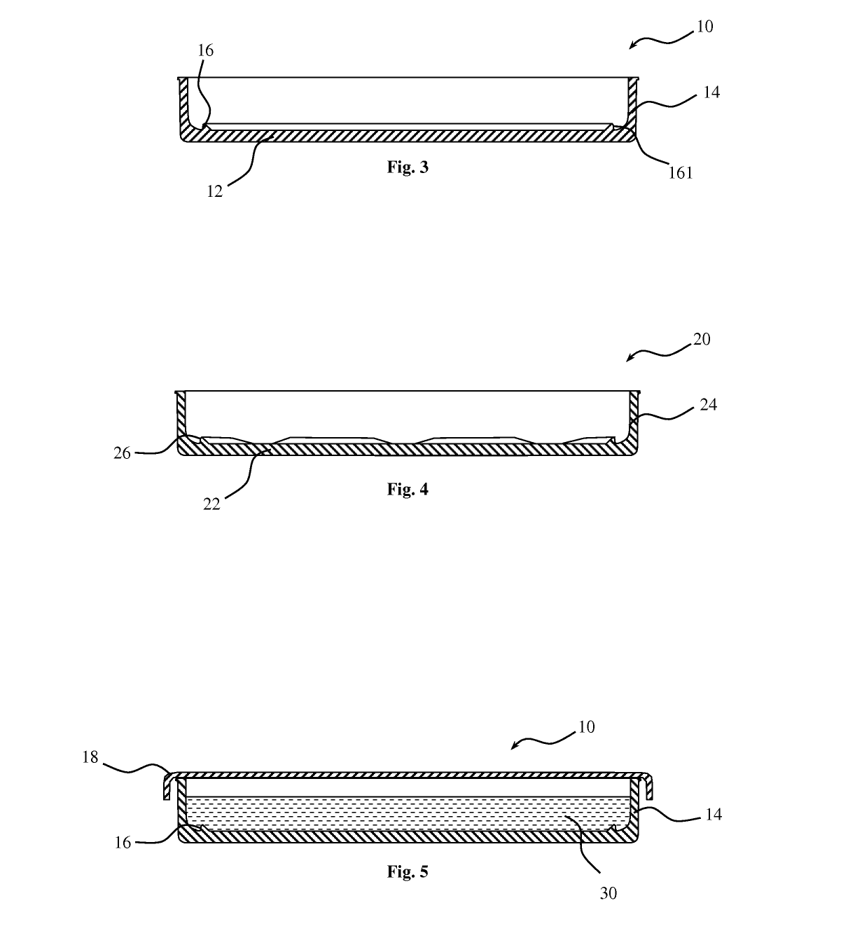

FIG. 3 shows a cross-sectional view of the base of the Petri dish according to the first embodiment.

FIG. 4 shows a cross-sectional view of the base of the Petri dish according to the second embodiment.

FIG. 5 shows a cross-sectional view of the Petri dish according to the invention, containing the agar culture medium.

limiting which follows, with reference to the drawings, in which

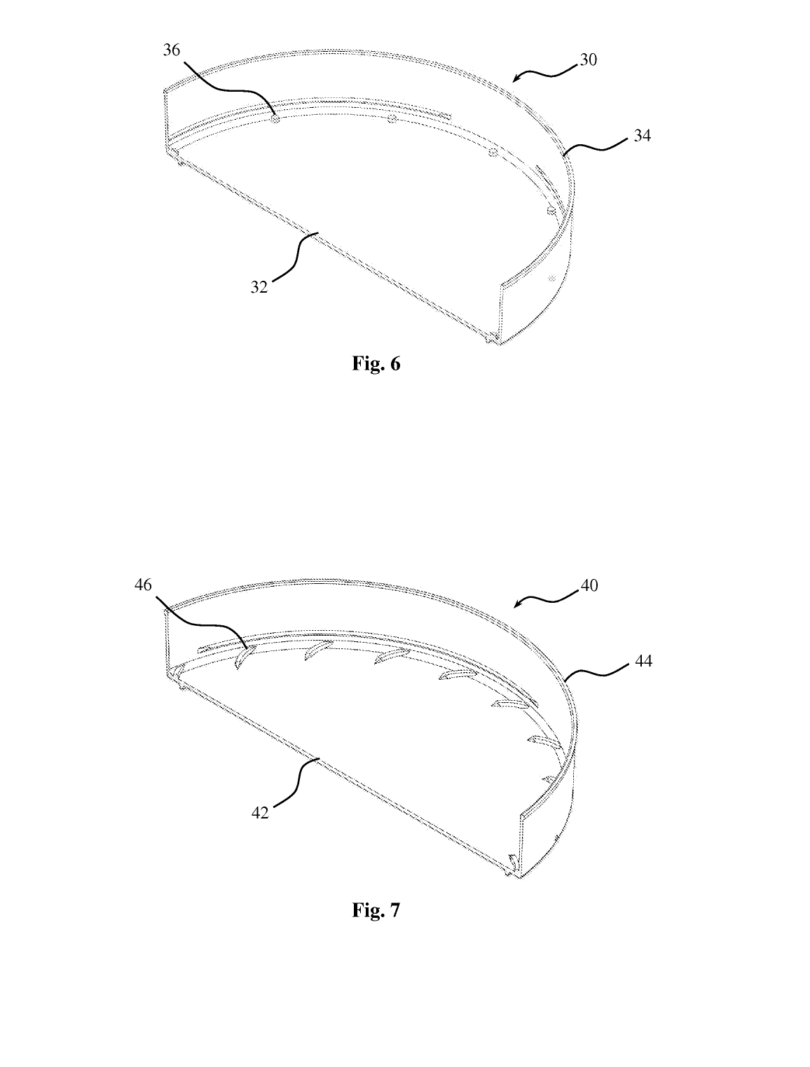

FIG. 6 shows a partial perspective view of the base of the Petri dish according to a third embodiment.

FIG. 7 shows a partial perspective view of the base of the Petri dish according to a fourth embodiment.

The Petri dish according to the invention conventionally has a base and a lid. In FIG. 1, there can be seen the base 10 of a Petri dish according to a first embodiment. This base 10 is constituted of a substantially flat and round bottom 12, and a circular vertical wall 14. The base 10, like the lid of the Petri dish, which is not shown, is made of plastic material conventionally used for this type of product, such as polystyrene. According to this first embodiment, the bottom 12 of the base 10 further comprises a continuous circular rib 16 which projects and runs along the circular vertical wall 14. The role of this rib 16 is to retain the agar culture medium arranged in a layer in the Petri dish. The rib 16 therefore acts as an anchoring point of the culture medium 30. This is clearly shown in FIG. 5.

As can be seen in detail in FIG. 3, the circular rib 16 has a cross-section which has a substantially triangular form. In particular, it is noted that the sides of the triangle forming the slopes of the rib do not have the same gradient. Indeed, it is altogether advisable that the slope 161 has a steep gradient facing the circular vertical wall 14. As explained above, the rib 16 serves as an anchoring point for the culture medium. Indeed, if in addition it can prevent the medium from detaching from the bottom, this anchoring has the task of preventing said culture medium from retracting. The retraction generally manifests itself in a reduction of the diameter of the culture medium layer, such that the edge of the culture medium layer is no longer in contact with the internal face of the circular vertical wall 14. The steep gradient of the slope 161 increases the rib's 16 ability to resist the retraction. In another particular embodiment, it can actually be envisaged that the slope 161 be inverted, thus increasing the anchoring phenomenon.

The height of the rib 16 must be much lower than the depth of the culture medium layer. This is essential because an excessively fine layer of culture medium in line with the rib 16 would generate at this place a weakness zone of the medium, at which the latter would be liable to break in the case of excessively strong and/or repeated mechanical stresses. Moreover, the rib must not impede the seeding of the culture medium. A rib of which the upper end is close to the surface of the culture medium could present such a disadvantage, without counting the risk of breaking, explained above, during the application of a seeding tool, such as an oese. All of these disadvantages of an excessively high rib should be balanced with the disadvantages of a rib which is too low in height, such that it no longer plays its anchoring role.

It is also preferable that the height of the rib 16 is preferably between 0.15 and 5 millimetres (mm). It should be understood that the height value is chosen in line with the shape of the cross-section of the rib. In other words, when it is desired to have a rib of lower height, the decrease in the retention ability of a rib of such a height can be compensated by a rib shape which improves the retention, such as a rib having an inverted slope 161.

FIGS. 2 and 4 show the base 20 of a Petri dish according to a second embodiment. This base 20 comprises like the base 10, a substantially flat and round bottom 22 as well as a circular vertical wall 24. In contrast, it is distinguished from the base 10 shown in FIGS. 1 and 3 due to the fact that it does not comprise one single continuous rib, but several non-continuous ribs 26, in arcs of circle, running along the circular vertical wall 14. These ribs are spaced apart from one another by an angle (constant or not) of between 10 and 170°. It should be noted that the distance between the ribs 26 taken in twos can be variable. Concerning the length of the ribs, it corresponds to an angle between 10 and 170°. Thus, for a Petri dish of 90 mm in diameter, this distance is between 7 mm and 13 cm. Likewise, the length of the non-continuous ribs within the one Petri dish can vary, from one rib to the other.

As can be seen in FIG. 4, these non-continuous ribs 26 have a cross-section in a shape which is substantially identical to that of the rib 16, namely substantially triangular. It can be seen in FIG. 2 that the non-continuous ribs 26 present arrow-pointed extremities. Indeed, the height of the ribs 26 decreases progressively until the rib disappears. In other words, the three vertices of the ribs 26 converge at each of the extremities of these latter. It should nevertheless be noted that the form of said extremities only constitutes one particular embodiment and that it is quite possible to envisage ribs which have different forms.

The base 20 such as presented in FIGS. 2 and 4 comprises a total of 6 ribs. It is nevertheless envisageable to have a base comprising fewer or more non-continuous ribs. Indeed, the base can comprise, for example, two non-continuous ribs, in which case said ribs will be of more substantial length. The base can comprise, for example, 10 ribs, in which case these latter have a less substantial length than those of the embodiment presented in FIGS. 2 and 4. In any case, the important thing is that the total length of the entirety of non-continuous ribs 26 is at least equal to, or even greater than half of the circumference of the circle, on the line of which said non-continuous ribs 26 are placed.

The distance at which the ribs 16 or 26 are from the circular vertical wall 14 or 24 respectively is also an important element to be taken into consideration. Indeed, the positioning of the ribs abides by several requirements:
firstly, the ribs must be positioned such that they do not impede the visual analysis of the seeded Petri dish. Indeed, the identification of the microorganisms undergoes a visual analysis of the colonies which have grown on the agar culture medium, which give the microbiologist important data in terms of orientation of the analysis. The presence of the ribs therefore must above all not constitute a hindrance to such visual analysis;
secondly, the ribs must be positioned such that they play an effective role against the retraction of the agar. To this end, they must not be either too far from the circular vertical wall, in which case their action is limited; nor too close to the circular vertical wall, in which case the culture medium is rendered fragile since the quantity of material between the ribs and the circular vertical wall is too small, causing a risk of the agar culture medium rupturing.

In addition, advantageously, the distance between a rib and the circular vertical wall must advantageously be between 0.5 and 15 mm.

FIG. 6 shows the base 30 of a Petri dish according to a third embodiment. This base 30 comprises a substantially flat and round bottom 32 as well as a circular vertical wall 34. In contrast, it differs from the two preceding embodiments shown respectively in FIGS. 1 and 3 and FIGS. 2 and 4, by the fact that they do not comprise ribs on the bottom 32, but rather cylindrical studs 36. These studs are spaced apart from one another by an angle (constant or not) of between 10 and 170°. It should be noted that the distance between the studs 36, taken in twos, can also be variable.

For its part, FIG. 7 shows a fourth embodiment in the form of a base 40 of a Petri dish. This base 40 comprises a substantially flat and round bottom 42 as well as a circular vertical wall 44. Ribs are arranged in arcs of circle 46 on the bottom 42. These ribs 46 are oriented substantially radially. These studs are spaced apart from one another by an angle (constant or not) of between 10 and 170°. It should be noted that the distance between the studs 36 taken in twos can be variable. The number and spacing of the ribs can vary according to the variants of this embodiment. By way of example, it is possible to envisage arranging a number of ribs between 4 and 20.

According to another particular embodiment which is not shown, the base of the Petri dish comprises several series of concentric ribs. These can be, independently from one series of ribs to the other, continuous or non-continuous ribs. Thus it is possible to envisage having several series of continuous ribs, several sets of non-continuous ribs or a combination of continuous and non-continuous ribs. In the event that the base of the Petri dish comprises several series of non-continuous ribs, these latter can be of identical or different lengths within a series or between different series. Furthermore, the non-continuous ribs can be aligned radially or offset relative to one another.

According to a last embodiment, the general form of the ribs can change. Indeed, the ribs according to the embodiments presented on the figures have a general circular shape for the continuous ribs or in arcs of circle for the non-continuous ribs. Nevertheless, it would be possible to have ribs in the form of segments of straight line.

It is also possible to envisage a continuous rib which has the general shape of a sinusoid following the circular vertical wall.

### EXAMPLE

### Measurement of Retraction of Agar Culture Media in Petri Dishes Comprising Means Intended to Avoid the Retraction and Conventional Petri Dishes

Measurements of retraction of an agar culture medium were performed with two different types of Petri dish: a conventional Petri dish and a Petri dish comprising means which make it possible to avoid retraction of the agar culture medium. The said means are constituted by a series of 6 non-continuous ribs; the ribs being aligned radially.

The protocol for the preparation of the agar culture medium and distributing this latter into the dishes is identical for all Petri dishes. Two different incubation profiles were tested for each type of dish.

### Test No. 1:

Production of the Agar Culture Medium and Distribution into the Petri Dishes:

The culture medium used is a bioMérieux ChromID® Ottaviani Agosti (Ref. 43641) medium.

16.5 kg of this medium in a dry form are diluted in water to obtain 228 litres (L) of culture medium. 720 conventional Petri dishes and 720 dishes with ribs are thus produced.

Storage/Incubation Conditions of the Petri Dishes:

The conventional Petri dishes and the Petri dishes with ribs produced as set out above are incubated under the following conditions:
Actual storage conditions: 10-15° C. for 4 days (logistical storage chamber), and then 2-8° C.
Conventional incubation conditions: 33-37° C. for 2 days (oven)

### Test No. 2

Production of the Agar Culture Medium and Distribution into the Petri Dishes:

The same quantity of bioMérieux ChromID® Ottaviani Agosti (Ref. 43641) medium is produced. 620 conventional Petri dishes and 620 dishes with ribs are thus prepared.

Storage/Incubation Conditions of the Petri Dishes:

The conventional Petri dishes and the Petri dishes with ribs produced as set out above were incubated under the following conditions:
Actual storage conditions: 10-15° C. for 5 days (logistical storage chamber)
Conventional incubation conditions: 33-37° C. for 2 days (oven)

For each of the Petri dishes produced in the two tests above, a visual analysis of the retraction of the agar culture medium in the dishes after storage/incubation is carried out. Table 1 below sets out all of the results:

TABLE 1

| TESTS | Test No. 1 | Test No. 2 |
|---|---|---|
| Conventional Petri | 124 dishes retracted out | 120 dishes retracted out |
| dish | of 720, or 17.2% | of 620, or 19.3% |
| Petri dish with | 2 dishes retracted out | No retraction |
| ribs | of 720, or 0.28% | |

The 2 tests presented above very clearly show that the ribs present in the Petri dishes greatly reduce or even eliminate the phenomenon of retraction of the agar culture medium contained in the dishes, regardless of the storage or incubation conditions.

1. Petri dish, intended for receiving an agar culture medium for microorganisms, comprising a base receiving said culture medium and a lid capable of being positioned on the base, said base and said lid being substantially circular, said Petri dish being characterised in that the base includes at least one means intended to avoid the retraction of the culture medium, said means projecting and being positioned on the periphery of the base.

2. Petri dish according to claim 1 wherein, the means intended to avoid the retraction of the culture medium is constituted by at least one continuous rib.

3. Petri dish according to claim 2, wherein the continuous rib is circular or sinusoidal.

4. Petri dish according to claim 1 wherein the means intended to avoid the retraction of the culture medium is constituted by at least one non-continuous rib.

5. Petri dish according to claim 1, further including means for locking the lid onto the base.

6. Petri dish according to claim 5, wherein the locking means include at least one radial fastening hook and at least one radial sheath, which are respectively positioned either on the lid, or on the base, and cooperate to lock the Petri dish.

7. Petri dish according to claim 4, including between 2 and 12 non-continuous ribs.

8. Petri dish according to claim 2, including several series of concentric, continuous and/or non-continuous ribs.

9. Petri dish according to claim 1, wherein the at least one means intended to avoid the retraction of the culture medium has a height of between 0.15 and 5 mm.

10. Petri dish according to claim 1, wherein the at least one means intended to avoid the retraction of the culture medium is situated at a distance of between 0.5 and 15 millimetres from the circular vertical edge of the base.

11. Method of preparing an agar culture medium comprising the steps consisting of:
possessing a Petri dish, according to claim 1,
distributing a molten agar culture medium inside said Petri dish, such that it covers the entire bottom of the base,
allowing the agar culture medium to cool such that the at least one means intended to avoid the retraction are anchored in the agar culture medium.
